(19) United States
(12) Patent Application Publication
(10) Pub. No.: US 2009/0099437 A1
(43) Pub. Date: Apr. 16, 2009

(54) Lancing Depth Adjustment Via Moving Cap

(76) Inventor: Vadim Yuzhakov, Alameda, CA (US)

Correspondence Address: SF Bay Area Patents, LLC;Attn: Andrew V. Smith, Ph.D., 601 Van Ness Avenue, #1108, San Francisco, CA, 94102, US

(21) Appl. No.: 11/870,420

(22) Filed: Oct. 11, 2007

(51) Int. Cl. A61B 5/157; A61B 5/151

(52) U.S. Cl. 600/365; 606/181; 600/583

(57) ABSTRACT

A lancing device includes a housing for containing a lancet. A lancet driver couples with and moves the lancet within the housing. A skin interface cap has an opening defined therein to expose at least part of the lancet when moved by the driver. A sliding frame is coupled to the housing and to the skin interface cap. The frame being adjustable relative to the housing for accordingly adjusting said cap relative to the housing, whereby an extent to which the lancet is exposed through the opening is adjustable by adjusting the sliding frame.

### BACKGROUND

Measurements of blood glucose levels typically involve a blood expression process for acquiring a sample of blood or other body fluid for analysis. To acquire a blood sample, for example, the skin may be punctured to a pre-determined depth using a needle or a lancet to create a small wound. Creation of a shallower wound is normally less painful than creation of a deeper wound. However, deeper wounds normally produce more blood than shallower wounds. Only if there is a sufficient amount of blood available will the measurement be possible and/or reliable depending on the type of assay being performed and on the assay equipment being used. Individuals differ in the amount of blood that is made available upon creation of a certain depth or extent of wound. Various individuals have different blood circulation, skin texture, etc., and would like to adjust the lancing depth to a comfortable yet useful value for them. It is desired to be able to adjust the lancing depth to an optimum value that is sufficient for producing enough blood for performing analysis and that is minimally painful.

When a lancing device, including an in integrated blood glucose monitoring system, is used, e.g., as described in U.S. application Ser. Nos. 11/535,985 and 11/585,986, it is also desired to be able to adjust the lancing depth adjustment. The lancing depth adjustment is more challenging for implementing in integrated blood glucose systems though, because such systems are already very complicated even without providing an additional lancing depth adjustment capability.

Some conventional stand-alone lancing devices include lancing depth adjustment capability. These are generally provided in one of two ways. The first way includes lancing depth adjustment which forms a part of the lancing mechanism. The second includes a rotatable, telescopic cap on the housing of the lancing device. Both of these approaches have significant disadvantages when used in integrated blood glucose monitoring systems. The first approach involves an additional complication of an already complex lancing-advance mechanism. The rotatable, telescopic cap involves incorporation of quite complicated telescopic mechanism on a compact blood expression cap.

### SUMMARY OF THE INVENTION

It is recognized by the present inventor that it would be advantageous to have a depth adjustment mechanism that is separate from a lancing mechanism, particularly in an integrated lancing and glucose sensing device, e.g., instead forming part of the external housing of the integrated device with the depth adjustment mechanism. Embodiments are described below that allow lancing depth adjustment via moving a part of the external housing to a predetermined distance with respect to the lancing mechanism.

Lancing devices are provided including a lancet for piercing skin and exposing blood to perform an assay. Embodiments include a lancet driver coupled with and moving a lancet within a housing. A skin interface cap has an opening defined therein to expose at least part of the lancet when moved by the driver. A sliding frame is coupled to the housing and to the skin interface cap. The frame is adjustable relative to the housing for accordingly adjusting the cap relative to the housing, whereby an extent to which the lancet is exposed through the opening is adjustable by adjusting the sliding frame.

Embodiments also include a housing for containing a lancet. A lancet driver couples with and moves the lancet within the housing. A sliding skin interface cap couples to the housing and has an opening defined therein to expose at least part of the lancet when moved by the driver. The sliding skin interface cap is adjustable relative to the housing for accordingly adjusting the opening relative to the housing, whereby an extent to which the lancet is exposed through the opening is adjustable by adjusting the sliding skin interface cap.

With regard to either of these lancing device embodiments or alternative modification thereof, the skin interface cap may be coupled at a hinge to the housing, whereby the skin interface cap pivots around the hinge when the sliding frame is adjusted relative to the housing. A depth adjustment knob may be coupled to the sliding frame or sliding skin interface cap for controlling the adjusting of the sliding frame or interface cap.

In certain embodiments, a glucose sensor and/or meter may also be disposed within the housing providing an integrated system for performing the assay upon application of blood to the sensor. The lancet driver may be configured to couple with the sensor such that upon retraction of the lancet, the lancet driver moves the sensor through the opening to contact and receive at least a portion of the blood.

Embodiments may also include a pressure sensor may be provided for triggering the lancet driver to move the lancet through the opening when a predetermined pressure is applied to the skin interface cap and/or for permitting lancing upon activation of a switch when a predetermined pressure is applied to the skin interface cap.

The cap may be shaped to promote blood expression at the opening.

Methods of lancing skin and exposing blood to perform an assay are also provided. In certain embodiments, a lancet penetration depth is adjusted by sliding a skin interface cap relative to a housing and/or selected by adjusting a sliding frame relative to the housing, and thereby moving the opening relative to a lancet or lancet driver or both. The lancet is coupled to and moved by the lancet driver. At least part of the lancet is exposed through the opening in the skin interface cap to pierce skin that is provided at the opening at a depth selected by the adjusting to the skin interface cap.

The methods may further include promoting expression of blood at the opening upon compression of the skin to the skin interface cap over the opening.

The adjusting of the lancing penetration depth by sliding the skin interface cap may include rotating a depth adjustment knob, or the like.

The methods may include rotating a depth adjustment knob for controlling the sliding of the skin interface cap.

The methods may also include retracting the lancet, coupling a sensor with the lancet driver, and moving the sensor through the opening by moving the lancet driver so that the sensor contacts and receives at least a portion of the blood.

The methods may include sensing a pressure applied to the skin interface cap and triggering the lancet driver to move the lancet through the opening, permitting lancing by activation of a switch and/or otherwise permitting lancing when a predetermined pressure is applied.

### BRIEF DESCRIPTION OF THE DRAWINGS

FIG. 1A schematically illustrates an external view of an integrated lancing and glucose monitoring device having lancing depth adjustment in accordance with a first embodiment, wherein the lancing depth is adjusted to a deepest position with the skin interface cap in contact with the fixed portion of the external housing.

FIG. 1B schematically illustrates an internal cross-sectional view of the device of FIG. 1A including a sliding frame for adjusting lancing depth by moving a skin interface cap relative to both a final most-exposed lancet position and the fixed portion of the external housing.

FIG. 1C schematically illustrates another external view of the device of FIG. 1A, wherein the lancing depth has been adjusted to a shallower depth by moving the cap away from the fixed portion of the housing, such that a small portion of the sliding frame is now visible from the outside.

FIG. 2A schematically illustrates an external view of an integrated lancing and glucose monitoring device having lancing depth adjustment in accordance with a second embodiment.

FIGS. 2B-2C include cross-section internal views of the device of FIG. 2A, particularly illustrating selected internal components.

FIG. 3 illustrates a skin interface surface of a blood expression cap in accordance with certain embodiments.

### DETAILED DESCRIPTION OF THE EMBODIMENTS

Lancing depth-adjustment features are provided for a lancing device and/or a fully (or partially) integrated blood glucose monitor that includes a lancing device and a meter for measuring a glucose level in a bodily fluid and/or otherwise determining a blood glucose level of a user. The depth adjustment is achieved by moving a surface of the fully integrated meter or lancing device to a pre-determined distance with respect to the lancing mechanism. The lancing mechanism is not affected with regard to its position or motion within the housing of the monitor. Instead, the position of a skin interface cap is adjusted relative to the housing and lancing mechanism, thereby adjusting the position of the skin of the user relative to the position of maximum exposure of the lancet out of the housing. That is, depth adjustment features are provided at the external housing of the lancing device or integrated system.

Embodiments of the skin interface caps include a blood expression cap for optimizing the obtaining of blood at a wound created by an incision formed by a lancing mechanism penetrating the skin of the user. The blood expression cap is designed to facilitate the expression of blood at the wound based on its contour or other physical design feature. The described design is mechanically robust and allows the application of a substantial load to the cap during the blood expression process. Also, the blood expression cap can be easily replaced or removed for cleaning purposes.

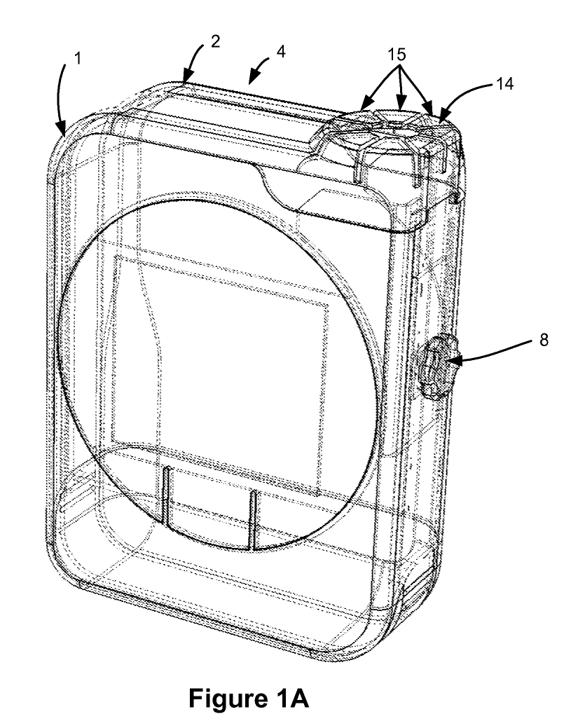

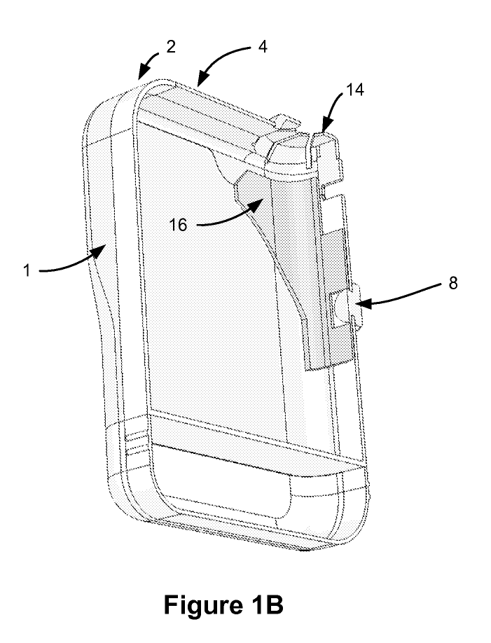

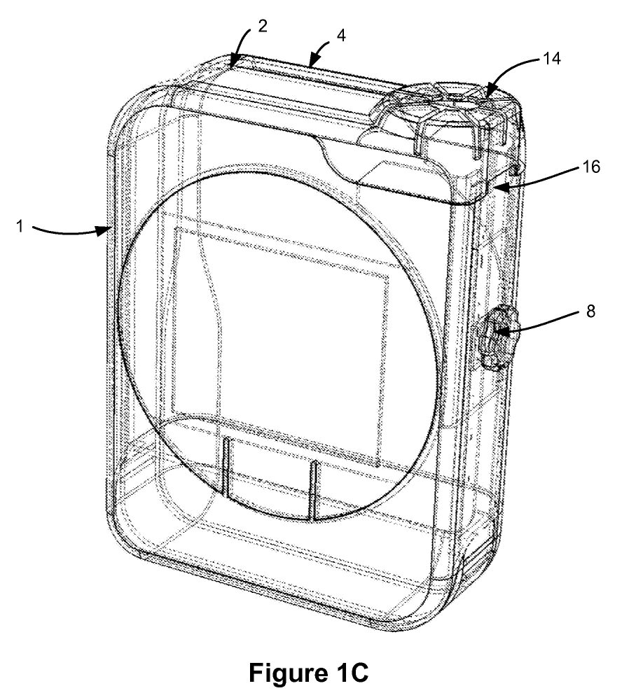

The penetration depth of the lancet into the skin tissue is adjusted by moving a feature on the external housing of the lancing device or the meter. FIG. 1A schematically illustrates an example in accordance with one of several possible embodiments of the moving lancing depth-adjustment mechanism. FIG. 1A shows a lancing device or integrated lancing and blood glucose measurement system including a housing 1 containing one or more lancets and a lancet driver. Several lancets or striplets each including a lancet and a sensor may be provided in a cartridge such as described in the priority applications incorporated by reference herein.

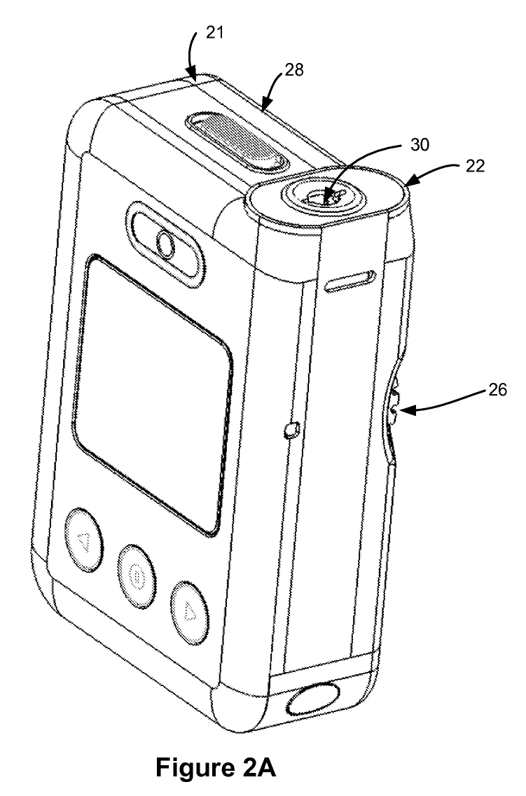

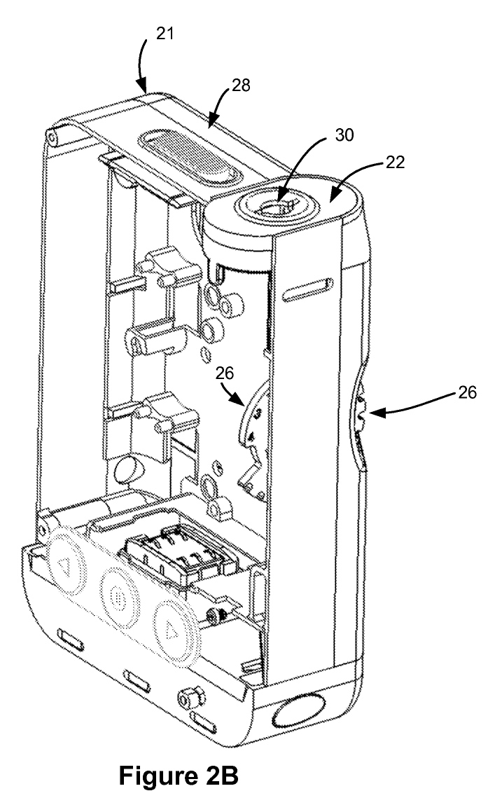

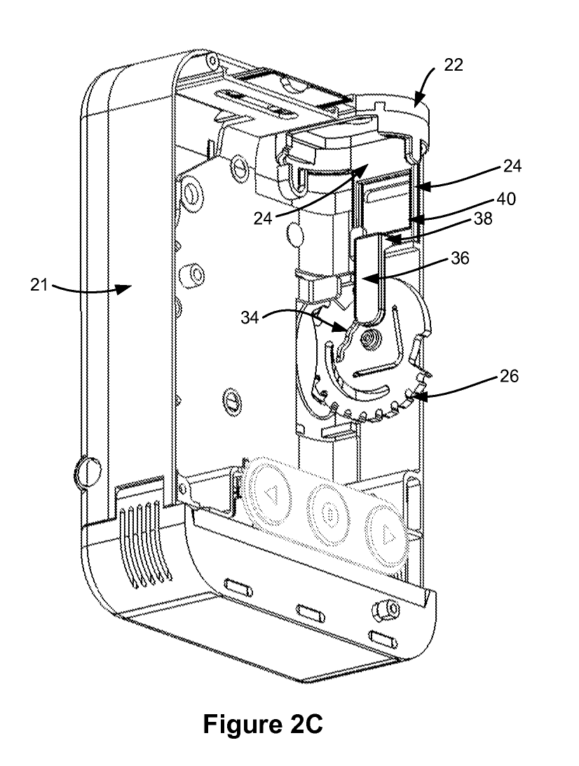

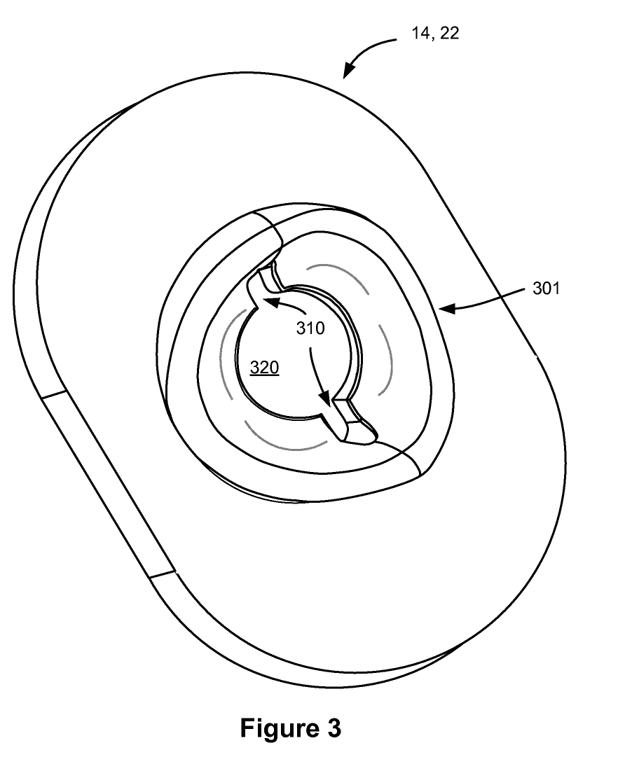

A skin interface cap 4 (“4” seems to be pointing to the end and not a “cap”) is in this case a blood expression cap 4 is mounted to the housing 1 at a hinge 2. Movement of the cap 4 is facilitated in the example of FIG. 1A by a depth adjustment knob 8. A sliding frame (not shown in FIG. 1A, but see FIGS. 1B-1C) contacts the blood expression cap 4 opposite the hinge 2 causing the cap 4 to rotate about the hinge by moving the sliding frame up or down. This effectively moves the skin interface portion 14 of the cap 4 relative to the rest of the housing 1 (see, e.g., United States published patent application no. 2006/0089566, which is hereby incorporated by reference. As the lancet is configured to protrude from the skin interface portion 14 of the cap 4 during a lancing process to a maximum protrusion position relative to the housing 1, upward adjustment (with respect to the illustration of FIG. 1A) of the portion 14 will result in a shallower wound and downward adjustment of the portion 14 will result in a deeper wound. Referring briefly to FIG. 3, the skin interface portion 14 may include a contoured section 301. The skin interface portion 14 may include a single contoured section 301, or may have multiple fingers 15 as in the embodiment illustrated at FIG. 1A. In addition, a slit 310 may be included which intersects an otherwise circular or elliptical opening 320, wherein the slit 310 permits test strips to protrude through the opening 320. It is noted that any of the embodiments described herein may include a contoured section 301, or a flat skin interface 14, 22, and the skin interface 14, 22 may include multiple fingers (FIG. 1A) or a single continuous section (FIGS. 2A and 3).

The mechanism illustrated at FIG. 1A uses the blood expression cap 4 which pivots around the hinge 2 located on the meter housing 1. Another side of the blood expression cap 4, where the skin interface portion 14 is located, is coupled to the sliding frame (see FIGS. 1B-1C) which moves internally within and relative to the fixed housing 1.

FIG. 1B is a cross-sectional internal view of the device of FIG. 1A. The fixed housing 1, hinge 2, blood expression cap 4 or skin interface cap 4 including contact portion 14 and depth adjustment knob 8 are also shown in FIG. 1A. The sliding frame 16 is not visible from the outside in FIG. 1A. The sliding frame 16 is, however, partially visible in the external view of FIG. 1C. The reason is that the depth adjustment is set at the deepest penetration depth in FIG. 1A, which is when the skin interface cap 14 is flush in contact with the fixed frame 1. The depth adjustment is set to a shallower depth in FIG. 1C, wherein the skin interface cap 4 has been moved away from the fixed frame 1 at the skin contact portion 14 through the action of the sliding frame 16. Because of the movement away from the fixed housing 1 of the portion 14, the sliding frame 16 is partially visible from the outside of the housing, as indicated in FIG. 1C.

When the sliding frame 6 is moved up or down, the skin interface portion 14 of the blood-expression cap 4 is correspondingly moved up or down as well. However, another end of the blood expression cap 4 is attached to the hinge 2 and does not move. Therefore, the cap 4 rotates around the axis of the hinge 2 as a result of the movement of the sliding frame 6 as controlled by the depth adjustment knob 8. This rotating motion tilts the cap 4 and raises or lowers the skin-interfacing portion 14 with respect to the lancing mechanism contained within the housing 1.

It is recognized in this embodiment that when the cap 4 is rotated, this will produce a small angular change between the normal to the skin interface portion 14 and the direction in which the moving lancet is pointed. That is, the angle with respect to the lancing mechanism between the skin-interfacing surface 14 of the blood expression cap 4 and the lancing direction will change, but such distance with respect to the lancing mechanism will be low as long as the hinge is located away from the lancing axis as illustrated at FIG. 1A, because the adjustments in depth are sufficiently small.

Another embodiment is illustrated schematically at FIGS. 2A-2C. Referring to FIG. 2A, the housing 21 of the lancing device or integrated lancing device and blood glucose monitor contains one or more lancets or striplets, a lancing driver, and perhaps glucose measurement electronics. In certain embodiments, a blood expression cap 22 includes a skin interface surface 30 that is shaped to facilitate the expression of blood and is coupled with a sliding frame (not shown in FIGS. 2A-2B, but see FIG. 2C or FIGS. 1B-1C) that is adjustable relative to fixed portion of the housing 21 and the lancing driver (also not shown) by knob 26. The position of maximum exposure of the lancet relative to the fixed housing is fixed, and so the lancing depth is adjustable by moving the skin interface surface 22 relative to the fixed housing 21. The cap 22 in this case includes only the end portion appearing as an oval shape in FIG. 2. The top 28 of the housing 21 does not move relative to the rest of the housing 21 when the skin interface cap 22 is moved in this embodiment.

FIGS. 2B-2C illustrate features of the depth adjustment mechanism that are internal to the housing 21 of the device shown in an external view in FIG. 2A. Referring specifically to FIG. 2C, the depth adjustment knob 26 is a cam 26 having a cam path 34 defined in it. A cam follower 36 includes a cam extension (not shown) which fits in the cam path 34. Rotation of the know 26 causes the cam follower 36 to move within the cam path 34 such that the cam follower moves relative to the substantial portion of the housing 21. Although not shown, a lancet driver remains fixed in its position relative to the housing 21 when the depth adjustment mechanism is manipulated. However, the blood expression cap 22 moves up and down with the movement of the follower 36, as a platform portion 38 of the cam follower 36 is coupled to, is in contact with, or is otherwise bound to follow the movement of the platform 38.

The embodiment of FIGS. 2A-2C uses a blood expression cap 22 which is, in certain embodiments, coupled directly to the sliding frame 24 which moves along and relative to the housing 21. The position of the sliding frame 24 is regulated by the knob 26 which is coupled to the meter housing 21 for adjusting lancing depth.

When the sliding frame 24 is moved up or down, one end of the blood-expression cap 22 is correspondingly moved up or down as well relative to the fixed housing 21 and a maximally-exposed position of the lancet when moved by the lancet driver (not shown). This motion moves the cap 22 and raises or lowers the skin contact surface 30 of the blood expression cap 22 with respect to the lancing mechanism.

The sliding frame 24 of either of the second embodiment provides stable adjustability of the skin interface cap 22, even though the cap 22 is not stabilized to the housing 21 at a hinge such as in the first embodiment. The sliding frame 24 contacts the cap 22 either at multiple points around the cap 22 or continuously for a sufficient extent that the cap 22 remains stable and does not wobble even when the cap 22 adjusted for shallow lancing depths away from the fixed housing. The cap 22 may have one or more legs that slide each in a channel provided in the fixed housing 21 to provide stability to the cap 22 by restricting the ability of the leg to rock due to its constraint within the channel.

Certain embodiments can be practiced with either a simple lancing device or a completely integrated blood glucose meter. Different embodiments may be used to change the lancing depth to express blood from fingers, forearm, and alternative sites for blood glucose measurements, and/or on other analyte measurement applications.

The present invention is not limited to the embodiments described above herein, which may be amended or modified without departing from the scope of the present invention as set forth in the appended claims, and structural and functional equivalents thereof.

In methods that may be performed according to the embodiments herein and that may have been described above and/or claimed below, the operations have been described in selected typographical sequences. However, the sequences have been selected and so ordered for typographical convenience and are not intended to imply any particular order for performing the operations.

In addition, all references cited above herein, in addition to the background, summary of the invention and brief description of the drawings sections, and the drawings, are all hereby incorporated by reference into the detailed description of the embodiments as disclosing alternative embodiments and components.

I claim:

1. A lancing device, comprising:
(a) a housing containing a lancet;
(b) a lancet driver to couple with and move the lancet within the housing;
(c) a skin interface cap having an opening defined therein to expose at least part of the lancet when moved by the driver; and
(d) a sliding frame coupled to the housing and to the skin interface cap, said frame being adjustable relative to the housing for accordingly adjusting said cap relative to the housing, whereby an extent to which the lancet is exposed through the opening is adjustable by adjusting the sliding frame.

2. The device of claim 1, further comprising a depth adjustment knob coupled to the sliding frame for controlling the adjusting of the sliding frame.

3. The device of claim 1, further comprising a pressure sensor for triggering the lancet driver to move the lancet through the opening when a predetermined pressure is applied to the skin interface cap.

4. The device of claim 1, wherein the device comprises an integrated device.

5. The device of claim 1, further comprising a lancet.

6. The device of claim 5, wherein the skin interface cap is also coupled at a hinge to the housing, whereby the skin interface cap pivots around the hinge when the sliding frame is adjusted relative to the housing.

7. The device of claim 5, further comprising a depth adjustment knob coupled to the sliding frame for controlling the adjusting of the sliding frame.

8. The device of claim 5, further comprising a glucose sensor and meter within the housing for performing said assay upon application of said blood to the sensor.

9. The device of claim 5, further comprising a pressure sensor for permitting lancing upon activation of a switch when a predetermined pressure is applied to the skin interface cap.

10. A lancing device, comprising:
(a) a housing for containing a lancet;
(b) a lancet driver to couple with and move the lancet within the housing;
(c) a sliding skin interface cap coupled to the housing and having an opening defined therein to expose at least part of the lancet when moved by the driver;
(d) wherein said sliding skin interface cap is adjustable relative to the housing for accordingly adjusting said opening relative to the housing, whereby an extent to which the lancet is exposed through the opening is adjustable by adjusting the sliding skin interface cap.

11. The device of claim 10, further comprising a depth adjustment knob coupled to the sliding skin interface cap for controlling the adjusting of the sliding frame.

12. The device of claim 10, further comprising a glucose sensor and meter within the housing for performing said assay upon application of said blood to the sensor.

13. A method of lancing skin and exposing blood to perform an assay, comprising:
(a) providing a housing containing a lancet, a lancet driver, and a sliding skin interface cap coupled to the housing and having an opening defined therein;
(b) adjusting a lancet penetration depth by sliding the skin interface cap relative to the housing and thereby moving the opening relative to the lancet or lancet driver or both;
(c) coupling the lancet to the lancet driver;
(d) moving the lancet by moving the lancet driver;
(e) exposing at least part of the lancet through the opening in the skin interface cap to pierce skin that is provided at the opening at a depth selected by the sliding of the skin interface cap.

14. The method of claim 13, further comprising rotating a depth adjustment knob for controlling the sliding of the skin interface cap.

15. The method of claim 13, further comprising sensing a pressure applied to the skin interface cap and triggering the lancet driver to move the lancet through the opening when a predetermined pressure is applied.

16. The method of claim 13, further comprising sensing a pressure applied to the skin interface cap and permitting lancing upon activation of a switch when a predetermined pressure is applied.

17. A method of lancing skin and exposing blood to perform an assay, comprising:
(a) providing a housing containing a lancet, a lancet driver, a sliding frame coupled to the housing, and a skin interface cap coupled to the sliding frame and having an opening defined therein;
(b) selecting a lancet penetration depth by adjusting the sliding frame relative to the housing and thereby moving the opening relative to the lancet or lancet driver or both;
(c) coupling the lancet to the lancet driver;
(d) moving the lancet by moving the lancet driver;
(e) exposing at least part of the lancet through the opening in the skin interface cap to pierce skin that is provided at the opening at a depth selected by the adjusting of the sliding frame.

18. The method of claim 17, further comprising promoting expression of blood at the opening upon compression of the skin to the skin interface cap over the opening.

19. The method of claim 17, wherein the adjusting of the lancing penetration depth by sliding the skin interface cap further includes rotating a depth adjustment knob.

20. The method of claim 17, further comprising sensing a pressure applied to the skin interface cap and triggering the lancet driver to move the lancet through the opening when a predetermined pressure is applied.

21. The method of claim 17, further comprising sensing a pressure applied to the skin interface cap and permitting lancing upon activation of a switch when a predetermined pressure is applied.

22. A method of obtaining a biological fluid sample from an individual, the method comprising;
positioning a lancing device relative to a desired lancing site of an individual;
sliding an external frame of the lancing device relative to the device housing to adjust penetration depth; and
actuating the device to obtain a biological fluid sample from the site.

23. The method of claim 22, further comprising contacting an analyte sensor with the obtained fluid to perform an assay to determine analyte concentration.

24. The method of claim 22, wherein the sensor is disposed within the housing.

25. The method of claim 22, wherein the sensor and lancet are connected.
